(19) United States
(12) Patent Application Publication
(10) Pub. No.: US 2024/0180183 A1
(43) Pub. Date: Jun. 6, 2024

(54) LACTOSE REDUCED DAIRY POWDER

(71) Applicant: DSM IP ASSETS B.V., Heerlen (NL)

(72) Inventor: Petrus Jacobus Theodorus DEKKER, Echt (NL)

(21) Appl. No.: 17/905,201

(22) PCT Filed: Mar. 9, 2021
(86) PCT No.: PCT/EP2021/055888

§ 371 Date: Oct. 27, 2022

(30) Foreign Application Priority Data
Mar. 10, 2020 (EP) 20161956.6

(51) Int. Cl. A23C 9/12; A23C 9/16; C12N 9/10; C12N 9/38

(52) U.S. Cl. CPC A23C 9/1206; A23C 9/1216; A23C 9/16; C12N 9/1051; C12N 9/2471; C12Y 204/01; C12Y 302/01023

(57) ABSTRACT

The invention relates to the field of dry or powder dairy compositions. The present invention describes a method for producing a lactose reduced dairy powder comprising
(i) treating a lactose comprising milk-based substrate with a first enzyme having transgalactosylase activity and a second enzyme having lactase activity to obtain a lactose reduced and galacto-oligosaccharides (GOS) comprising milk-based product
(ii) preparing lactose reduced dairy powder from said lactose reduced and GOS comprising milk-based product.

### CROSS-REFERENCE TO RELATED APPLICATIONS

This application is the National Stage entry of International Application No. PCT/EP2021/055888, filed 09 Mar. 2021, which claims priority to European Patent Application No. 20161956.6, filed 10 Mar. 2020.

### REFERENCE TO SEQUENCE LISTING SUBMITTED AS A COMPLIANT ASCII TEXT FILE (.TXT)

Pursuant to the EFS-Web legal framework and 37 C.F.R. § 1.821-825 (see M.P.E.P. § 2442.03(a)), a Sequence Listing in the form of an ASCII-compliant text file (entitled “Sequence_Listing_2919208-591000_ST25.txt” created on 9 Feb. 2023, and 21,457 bytes in size) is submitted concurrently with the instant application, and the entire contents of the Sequence Listing are incorporated herein by reference.

### BACKGROUND

### Field

The invention relates to the field of dry or powder dairy compositions.

### Description of Related Art

Generally, dairy powders are produced to preserve the nutrition of dairy for longer periods of time. Yet another advantage of dairy powders is that their transportation costs are severely reduced when compared to fluid dairy.

Dairy powders can be produced from milk or whey that is made lactose-free via the batch process. In this process the milk or whey is treated with a lactase enzyme before the concentration and drying of the product. A major problem is the presence of a high concentration of monosaccharides in the treated liquid dairy, leading to a drop in the glass-transition temperature. Hence, this product will lead to fouling of the spray dryer when the drying conditions are not adapted [Torres et al., 2017, technological aspects of lactose-hydrolyzed milk powder, Food Research International 101, 45-53]. The much milder spray drying conditions required, dramatically decreases productivity of the drying process, and increases costs. Additionally, the lactose-free milk (or whey) powder is highly hygroscopic, leading to caking during storage when not packaged with extra caution. Due to these challenging technical problems, lactose-free dairy powders are still a small market, in contrast to regular milk powders.

EP0458358 B1 (Snow Brand) describes a process for producing skim milk powder containing 10-15% by weight of galacto-oligosaccharides (GOS) by concentrating skim milk and incubating the concentrated milk with beta-galactosidase, followed by enzyme inactivation and spray-drying. The produced product is not lactose-free.

WO2013/182686 (DuPont) describes a polypeptide having transgalactosylating activity which is used to produce GOS-comprising yogurt. The produced product is not lactose-free. WO2013/182686 does not relate to dairy powder.

WO 2015/086746 (DuPont) describes a method for producing a milk product containing a stable amount of GOS, where the product needs to be heat-treated after production to inactivate the enzyme. The produced product is not lactose-free. Dairy powder is not described.

WO2018/210821 (Novozymes) describes a method for producing a milk product starting from a milk substrate comprising at least 20 wt % lactose and contacting the milk substrate with an enzyme having transgalactosylating activity. The produced product is not lactose-free.

WO2020/117548 (DuPont) describes a method for preparing a low lactose milk-based product having GOS fiber the method having the steps of providing a milk-based substrate comprising lactose; treating said milk-based substrate with a transgalactosylating enzyme to provide GOS fiber and remaining lactose; deactivating the transgalactosylating enzyme, contacting the milk-based substrate having GOS fiber with a lactase to degrade the remaining lactose to provide the low lactose milk-based product having GOS fiber; and deactivating the lactase.

The disadvantage of the prior art methods is that the transgalactosylating enzyme needs to be inactivated before the lactase is added and/or that the sequential use of the transgalactosylating enzyme and the lactase are time consuming and/or that the obtained product is not lactose free and/or that the milk substrate needs to comprise increased levels of lactose and/or that the prior art methods are complex and/or that spray drying of lactose free powder shows technical problems and/or that the produced GOS is broken down.

### SUMMARY

The aim of the present invention is to provide a method for producing a lactose reduced (preferably lactose free) dairy powder which, for example, does not dramatically decrease the productivity of the drying equipment, i.e. the invention aims at increasing the drying capacity and/or to decrease the production time, compared with the capacity and production time of the same drying equipment which is used to dry a comparable lactose reduced dairy powder which does not (or hardly not) comprise GOS. In addition or alternatively, the aim of the present invention is to produce lactose reduced (preferably lactose free) dairy powder which has improved characteristics, i.e. is less hygroscopic and/or less sticky, compared with the hygroscopicity and stickiness of a comparable lactose reduced dairy powder which does not or hardly not comprise GOS. In addition or alternatively, the present invention aims to reduce the mass loss in lactose reduced (preferably lactose free) milk powder production. In addition or alternatively, the present invention aims to reduce the brown color formation of lactose reduced (preferably lactose free) milk powder formed during drying or storage. In addition or alternatively, the present invention aims at improving, such as simplifying, prior art methods.

The invention provides

a method for producing a lactose reduced dairy powder comprising
(i) treating a lactose comprising milk-based substrate with a first enzyme having transgalactosylase activity and a second enzyme having lactase activity to obtain a lactose reduced and galacto-oligosaccharides (GOS) comprising milk-based product
(ii) preparing lactose reduced dairy powder from said lactose reduced and GOS comprising milk-based product,

lactose free dairy powder comprising at least 4% (w/w) GOS,

lactose free milk comprising at least 10% (w/w) GOS based on total carbohydrates and having a glucose/galactose ratio of >1.2,

milk comprising <0.1% lactose, at least 10% GOS based on total (free/) carbohydrates and having a glucose/galactose ratio of >1.2,

use of GOS for improving drying of a lactose reduced milk-based substrate,

use of an enzyme having transgalactosylase activity and an enzyme having lactase activity for improving the drying of a lactose free milk-based substrate, and

use of an enzyme having transgalactosylase activity and an enzyme having lactase activity for improving a characteristic of lactose free milk powder,

### FIGURES

FIG. 1: Diagram for production of lactose reduced/lactose free milk powder

### SEQUENCE LISTING

SEQ ID NO: 1 sets out the protein sequence of the β-hexosyltransferase of Sporobolomyces singularis (BHT). This sequence consist of a signal sequence of 22 amino acids and a deduced mature protein sequence of 572 amino acids. The amino acid sequence of BHT of Sporobolomyces singularis is also set out in http://www.uniprot.org/uniref/UniRef90_Q564N5. This sequence is also referred to as BHT-132 or wildtype polypeptide without any deletion.
SEQ ID NO: 2 sets out the sequence of BHT-134 which is a deletion mutant derived from SEQ ID NO: 1 and the deletion starts at position 18 up to and including position 54.
SEQ ID NO: 3 sets out a codon-adapted DNA sequence for expression of SEQ ID NO: 1 in Aspergillus niger.
SEQ ID NO: 4 sets out a codon-adapted DNA sequence for expression of SEQ ID NO: 2 in Aspergillus niger.
SEQ ID NO: 5 sets out the modified translational initiation sequence of the glucoamylase glaA promoter.
SEQ ID NO: 6 sets out the DNA fragments containing a.o. part of the glucoamylase promoter and the BHT-132 encoding gene including an EcoRI and PacI restriction site
SEQ ID NO: 7 sets out the DNA fragments containing a.o. part of the glucoamylase promoter and the BHT-134 encoding gene including an EcoRI and PacI restriction site

### DETAILED DESCRIPTION

In a first embodiment, the invention provides a method for producing a lactose reduced dairy powder comprising
(i) treating a lactose comprising milk-based substrate with a first enzyme having transgalactosylase activity and a second enzyme having lactase activity to obtain a lactose reduced and galacto-oligosaccharides (GOS) comprising milk-based product
(ii) preparing lactose reduced dairy powder from said lactose reduced and GOS comprising milk-based product.

Preferably, the invention provides a method for producing a lactose free dairy powder comprising
(i) treating a lactose comprising milk-based substrate with a first enzyme having transgalactosylase activity and a second enzyme having lactase activity to obtain a lactose free and galacto-oligosaccharides (GOS) comprising milk-based product
(ii) preparing lactose free dairy powder from said lactose free and GOS comprising milk-based product.

The phrases “a lactose reduced dairy powder” and “a dairy powder having a reduced amount of lactose” are used interchangeably herein.

The phrases “a lactose free dairy powder” and “a dairy powder free of lactose” are used interchangeably herein.

The phrases “lactose comprising milk-based substrate” and “milk-based substrate comprising lactose” are used interchangeably herein.

The phrases “a lactose reduced, galacto-oligosaccharides (GOS) comprising milk-based product” and “a lactose reduced and galacto-oligosaccharides (GOS) comprising milk-based product” and “a milk based product having a reduced amount of lactose and comprising galacto-oligosaccharides (GOS)” are used interchangeably herein.

The term “dairy powder” as used herein refers to a dry powder prepared from a milk-based product. The dairy powder can be milk or whey powder. Examples of milk powder are skim milk powder, semi-skimmed milk powder, full fat milk powder, butter milk powder, milk protein concentrate (MPC) powder, and examples of whey powder are sweet whey powder or acid whey powder, whey protein concentrate (WPC) powder, and whey permeate powder.

Preferably, the dairy powder is milk powder and more preferably, the dairy powder is lactose free milk powder and hence the invention provides a method for producing a lactose free milk powder comprising
(i) treating a lactose comprising milk-based substrate with a first enzyme having transgalactosylase activity and a second enzyme having lactase activity to obtain a lactose free and galacto-oligosaccharides (GOS) comprising milk-based product
(ii) preparing lactose free milk powder from said lactose free and GOS comprising milk-based product.

The term “milk” as used herein refers to the lacteal secretion obtained from any mammal, such as cows, sheep, goats, camels or buffaloes. Preferably, the milk is cow's milk.

The term “milk-based substrate” as used herein refers to any raw and/or processed milk material. The milk-based substrate may have been subjected to treatments known in the art such as—for example—homogenization, pasteurization, sterilization or an extended shelf life (ESL) treatment or an ultra-heat treatment (UHT).

The milk-based substrate may be skim milk, full fat milk, semi-skimmed milk, condensed milk, reconstituted (skim) milk, butter milk, milk protein concentrate (MPC), or whey such as sweet whey or acid whey, whey protein concentrate (WPC), whey protein isolate (WPI) or whey permeate.

Preferably, the milk-based substrate may be skim milk, full fat milk, semi-skimmed milk, condensed milk, reconstituted (skim) milk, butter milk or milk protein concentrate (MPC).

The milk-based substrate comprises lactose and hence the milk-based substrate is referred to as “a lactose comprising milk-based substrate”. In general, cow's milk comprises approximately 5% lactose. Preferably, the milk-based substrate only comprises the lactose which is naturally present in the milk-based substrate. Preferably, the milk-based substrate does not comprise added lactose. Preferably, the milk-based substrate comprises lactose in the range of 4 to 10 (w/v) % lactose, preferably 4 to 8 (w/v) % and even more preferably 4 to 6 (w/v) % or 4.5 to 5.5 (w/v) % lactose.

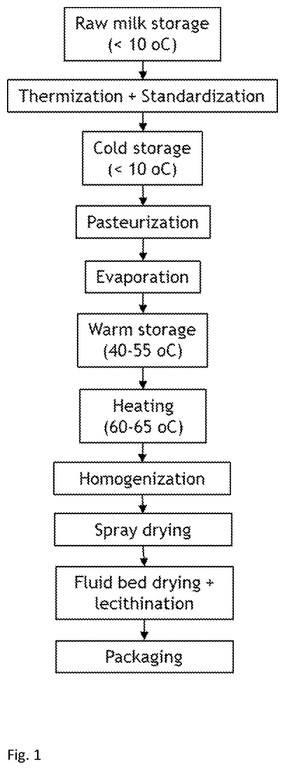

The milk-based substrate may be concentrated. The preparation of dairy powder typically comprise, as shown in FIG. 1, an evaporation step in which the substrate is concentrated. As will be explained later on, the enzymes used in a method of the invention, may be added at different points in time during the dairy powder production and could for example be added during or after evaporation in which case the milk-based substrate is concentrated. Preferably, the milk-based substrate is concentrated such that the amount of lactose is within the range of 4 to 10 (w/v) % lactose, preferably 4 to 8 (w/v) % and even more preferably 4 to 6 (w/v) % or 4.5 to 5.5 (w/v) % lactose.

The lactose comprising milk-based substrate is incubated with a first enzyme having transgalactosylase activity, i.e. an enzyme which produces GOS from lactose.

I.e. the GOS is produced in situ which means that the GOS is produced in the milk-based substrate from the lactose present in said milk-based substrate. Preferably, a method, use or product according to the invention does not comprises added GOS (i.e. non in situ produced GOS). Beta-galactosidases are enzymes which hydrolyse terminal non-reducing beta-D-galactose residues in beta-D-galactosides, for example lactose is hydrolysed to galactose and glucose. These enzymes belong to the enzyme class EC 3.2.1.23. Besides hydrolysing, this enzyme class is also able to transfer galactose to other sugars and thereby produce galacto-oligosaccharides (GOS). The different enzymes of class EC 3.2.1.23 have various preferences for hydrolytic (beta-galactosidase) activity and transgalactosylase activity and the preference can be expressed for example by the ratio of transgalactosylating activity to hydrolysing activity.

The first enzyme as used in a method of the invention is more transgalactosylating then hydrolysing. The first enzyme has predominantly transgalactosylating activity.

The enzyme having transgalactosylase activity preferably belongs to subclass EC 3.2.1.23.

As used herein, the terms “enzyme having transgalactosylase activity”, “enzyme having transgalactosylating activity”, “transgalactosylating enzyme” and “transgalactosylase” are used interchangeably herein and all refer to an enzyme capable of transferring galactose from lactose to a hydroxyl group of, for example, lactose, D-galactose (gal) or D-glucose (glu) whereby galacto-oligosaccharides are produced.

The activity of the first enzyme (i.e. the enzymatic activity leading to the production of GOS) is allowed sufficient time to produce a certain level of GOS molecules. The exact time will depend on the enzyme used, the amount of enzyme used but also on the used temperature and the lactose concentration in the milk-based substrate. The skilled person is capable to identify an enzyme dosage, incubation temperature and incubation time to allow the production of GOS in the milk-based substrate.

The lactose comprising milk-based substrate is incubated with the first enzyme having transgalactosylase activity such that sufficient GOS has been formed, i.e. at least 10% GOS based on total carbohydrates.

A suitable commercial example of a first enzyme having transgalactosylase activity is Zymstar™ GOS, or Nurica™ from DuPont or Biolacta FN5 from Amano. Many other enzymes having transgalactosylase activity are described in the scientific literature and/or in patent literature. Enzymes capable of transgalactosylation have been isolated from a wide range of micro-organisms, including bacteria, fungi and yeasts (Torres et al. (2010) Comprehensive Reviews in Food Science and Food Safety, 9: 438-454). GOS yield from lactose can vary depending on the origin of the enzyme, lactose concentration, pH, enzyme dosage, time and temperature of the incubation.

Another suitable example of an enzyme having transgalactosylase activity is a Sporobolomyces singularis hexosyltransferase, a Bacillus circulans beta-galactosidase or a Bifidobacterium bifidum beta-galactosidase.

Additionally, beta-galactosidases which are predominantly hydrolytic can be modified to behave more like a transgalactosylating enzyme by using the methods described in WO2018/210820.

Most suitable are enzymes with transgalactosylase activity that produce GOS species that are not easily degraded by the enzyme having lactase activity, for example an enzyme having transgalactosylase activity which is a Sporobolomyces singularis hexosyltransferase.

The second enzyme used in a method of the invention also belongs to subclass EC 3.2.1.23 and is also a beta-galactosidase but with a preference for lactase activity or hydrolysing activity.

The activity of the second enzyme (i.e. hydrolysis of lactose to galactose and glucose) is allowed sufficient time to reduce the amount of lactose. Preferably, the activity of the second enzyme results in a lactose reduced, more preferably lactose-free, milk-based product. The exact time will depend on the exact enzyme used, the amount of enzyme used but also on the used temperature. The skilled person is capable to identify an enzyme dosage, incubation temperature and incubation time to allow the hydrolysis of lactose in the milk-based substrate in galactose and glucose.

A suitable commercial example of an enzyme having hydrolytic activity on lactose is Maxilact (DSM), Lactozyme (Novozymes), Saphera (Novozymes), Nolafit (Chr. Hansen), Ha-lactase (Chr. Hansen), Godo YNL-2 (Dupont) and some other enzymes described as lactases. Other suitable lactases have been described in the scientific literature and/or in patent publications. Most preferably the enzyme having lactase activity has a preference for hydrolysing lactose, while the GOS species produced by the first enzyme having transgalactosylase activity remain intact.

As described above, beta-galactosidases can have transgalactosylase activity as well as lactase activity. The main activity depends for example on the lactose concentration in the milk-based substrate. Herein, an enzyme having transgalactosylase activity is an enzyme which (at the used conditions) has as its main activity, the production of GOS (but could have a minor lactase activity). An enzyme having lactase activity is an enzyme which (at the used conditions) has as its main activity, hydrolysis of lactose to glucose and galactose (but could have a minor activity which results in the production of GOS). Alternatively phrased, the first enzyme has predominantly transgalactosylase activity and the second enzyme has predominantly lactase activity. The skilled person is capable of selecting the right first and second enzymes. Suitable examples are provided above as well as in the examples.

The first and second enzyme are inactivated after incubation by any method known in the art which results in lowering of the enzyme activity. An example of a suitable inactivation method is a heat treatment, such as a pasteurization, or the heat treatment during the evaporation and concentration of the milk-based substrate, before spray drying. Preferably, the enzymes are completely inactivated (<1% of the initial activity remaining). Inactivation of the first enzyme may either be before the incubation with the second enzyme, but preferably at the same time as the second enzyme, after the incubation.

The used enzymes may be added as a liquid or as a solid (granulate) to the milk-based substrate.

The combined action of the first and second enzyme, result in a lactose reduced (preferably lactose free) and GOS comprising milk-based product which can be further processed into a lactose reduced (preferably lactose free) dairy powder.

A GOS can be a disaccharide (Degree of Polymerization (DP) 2, i.e. DP2) (except lactose which is not GOS), a trisaccharide (DP3), tetrasaccharide (DP4), pentasaccharide (DP5) or longer oligosaccharides.

An example of a disaccharide is galactosyl-galactose (gal-gal) or allolactose (galactosyl-beta 1-6-glucose). An example of a trisaccharide (DP3) is galactosyl-lactose (gal-gal-glu). An example of a tetrasaccharide (DP4) is galactosyl-galactosyl-lactose (gal-gal-gal-glu) An example of a pentasaccharide (DP5) is galactosyl-galactosyl-galactosyl-lactose (gal-gal-gal-gal-glu). An example of an even longer oligosaccharide is (galactose)n-lactose with n>3.

The term “DPₓ₊ GOS” means the sum of GOS molecules having DPₓ or higher DP. For example the term “DP3₊ GOS” means the sum of GOS molecules having DP3, DP4, DP5 etc.

The term GOS as used herein refers to the combination of DP2₊ GOS. DP2₊ GOS excludes lactose (which is by definition not GOS).

The level of GOS molecules can be determined by using different analysis techniques.

For example, one can determine the amount of lactose, glucose and galactose in the lactose comprising milk-based substrate and after incubation with the enzymes, determine the amount of lactose, glucose and galactose using an enzymatic analysis, NMR or HPAEC-PAD. Quantification of mono-, disaccharides can also be performed by using high-performance anion exchange chromatography with pulsed amperometric detection (HPAEC-PAD) analysis. In yet another alternative method, GOS production can be established by reaction of an enzyme on lactose in which the amount of galactose generated is less than the amount of glucose generated at a given time. The amount of GOS can then be determined by subtracting the sum of the amount of lactose, glucose and galactose (as present in the product) from the sum of the starting amount of lactose, glucose and galactose (as present in the substrate). The difference is then the amount of GOS. In a formula:

[GOS]_{product}=([lac]+[glu]+[gal])_{substrate}−([lac]+[glu]+[gla])_{product}

Determining the level of GOS with this formula can be performed in respect of a method of the invention, because the skilled person has the opportunity to determine the level of lactose in the milk-based substrate. Obviously, this determination/formula cannot be used if the level of lactose in the milk-based substrate cannot be determined, which is for example the case if one is provided with the lactose-reduced dairy powder (preferably lactose free milk powder) only. Another way to determine the level of GOS in a lactose-reduced dairy powder (preferably lactose free milk powder) will be provided later on.

The step of preparing lactose reduced (preferably lactose free) dairy powder from said lactose reduced (preferably lactose free), GOS comprising milk-based product, is performed using techniques which are well known in the art.

Typically, the dairy powder is made by evaporation and drying the produced lactose reduced, GOS comprising milk-based product to dryness. Drying is typically performed by spray drying, drum drying or freeze drying. Preferably the dairy powder is obtained by spray drying.

An example of a suitable scheme for producing a lactose reduced dairy powder is provided in FIG. 1. Preferably, the enzymes are added (at the same time or sequentially, preferably sequentially) when the milk is in cold storage. Such a storage is typically already present at a dairy powder plant and does not need additional equipment or changes to the dairy powder production process. Alternatively, the enzymes are added during or after evaporation.

As described above, the invention provides in a first embodiment, a method for producing a lactose reduced dairy powder comprising
(i) treating a lactose comprising milk-based substrate with a first enzyme having transgalactosylase activity and a second enzyme having lactase activity to obtain a lactose reduced and galacto-oligosaccharides (GOS) comprising milk-based product
(ii) preparing lactose reduced dairy powder from said lactose reduced and GOS comprising milk-based product.

Different surprising results have been noted by the inventors of the present patent application (when compared to the situation in which no, hardly no or low levels of GOS are present in lactose free milk), such as an increased glass transition temperature, an improved drying capacity of the (spray) dryer, a reduced loss of the (spray) dryer capacity and a reduction of the (spray) drying costs.
Hence, the invention alternatively provides

a method for improving (increasing) the drying capacity of a (spray) dryer for lactose reduced (preferably lactose free) milk powder

a method for improving (decreasing) the drying time for lactose reduced (preferably lactose free) milk powder

a method for improving (increasing) the drying speed for lactose reduced (preferably lactose free) milk powder

a method for improving (decreasing) the hygroscopic behaviour of lactose reduced (preferably lactose free) milk powder

a method for improving (decreasing) the stickiness of lactose reduced (preferably lactose free) milk powder

a method for improving (reducing) the brown color formation of lactose reduced (preferably lactose free) milk powder

wherein any of said method comprises
(i) treating a lactose comprising milk-based substrate with a first enzyme having transgalactosylase activity and a second enzyme having lactase activity to obtain a lactose reduced and galacto-oligosaccharides (GOS) comprising milk-based product
(ii) preparing lactose reduced dairy powder from said lactose reduced and GOS comprising milk-based product.

Another different surprising result is that a milk-based product could be prepared which comprises at least 10% (w/w) GOS (based on total carbohydrates) and is lactose-free. Hence, the invention also provides a method for preparing a milk-based product which comprises at least 10% (w/w) GOS (based on total carbohydrates) and is lactose free, said method comprising treating a lactose comprising milk-based substrate with a first enzyme having transgalactosylase activity and a second enzyme having lactase activity to obtain a lactose free and galacto-oligosaccharides (GOS) comprising milk-based product.

The already provided definitions, apply to these embodiments as well.

The (lactose reduced and GOS comprising) milk-based product as obtained by all the above described methods (as an intermediate product), comprises at least 10% GOS based on total carbohydrates. The term “total carbohydrates” in a milk-based substrate is defined as the sum of the concentrations of glucose, galactose, lactose and GOS.

As used herein, the term “lactose reduced and GOS comprising milk-based product” refers to a lactose reduction to 0.5 (w/v) % or 0.1 (w/v) %. The term “lactose free, GOS comprising milk-based product” is used to refer to a lactose reduction to <0.01% (w/v) (<0.1 g/L).

The term lactose free dairy powder (preferably lactose free milk powder) refers to a lactose reduction to <0.1% (w/w) (<1 g/kg).

Hence, the invention provides a method for producing a lactose reduced (preferably lactose free) dairy powder comprising
(i) treating a lactose comprising milk-based substrate with a first enzyme having transgalactosylase activity and a second enzyme having lactase activity to obtain a lactose reduced (preferably lactose free) and galacto-oligosaccharides (GOS) comprising milk-based product
(ii) preparing lactose reduced (preferably lactose free) dairy powder from said lactose reduced (preferably lactose free) and GOS comprising milk-based product,

wherein the lactose in said milk-based product is reduced to 0.5 (w/v) %, preferably 0.1 (w/v) % lactose, or

wherein the lactose in said milk-based product is reduced to <0.01 (w/v) % lactose and wherein the lactose reduced, GOS comprising milk-based product is a lactose free, GOS comprising milk-based product.

The invention thus provides a method for producing a lactose free dairy (preferably milk) powder comprising
(i) treating a lactose comprising milk-based substrate with a first enzyme having transgalactosylase activity and a second enzyme having lactase activity to obtain a lactose free and galacto-oligosaccharides (GOS) comprising milk-based product
(ii) preparing lactose free dairy (preferably milk) powder from said lactose free and GOS comprising milk-based product,

wherein the lactose in said lactose free, GOS comprising milk-based product is reduced to <0.01 (w/v) % lactose.

The GOS and lactose level in the corresponding powder will be different when compared to the level of these components in the obtained lactose reduced (preferably lactose free), GOS comprising milk-based product due to the concentration effect of the drying.

The GOS level in the dairy powder is at least 4% (w/w). Preferably, the level of GOS in the dairy powder is at least 5%, 6%, 7%, 8%, 9% or at least 10% (w/w). More preferably, the level of GOS in the dairy powder is at least 15% (w/w). The level of GOS in the dairy powder is not only dependent on the specific enzymes used but also on, for example, the fat percentage in the milk-based substrate.

The level/amount of lactose in a lactose reduced dairy powder is <5%, preferably <1% (w/w). The level of lactose in a lactose free dairy (preferably milk) powder is <0.1% lactose.

Examples of a first enzyme having transgalactosylase activity have been provide above. Yet another example of an enzyme having transgalactosylase activity is a Sporobolomyces singularis hexosyltransferase.

It is known that several basidiomycetous yeasts have potent transgalactosylation activities, and GOS production by Sporobolomyces singularis (formerly called Bullera singularis), Cryptococcus laurentii, Sterigmatomyces eleviae, Rhodotorula minuta, and Sirobasidium magnum has been reported. A major difficulty is producing these hexosyltransferases in sufficient amounts, making the commercial production of GOS with these enzymes inhibitory expensive. An obstacle in producing these enzymes is their cellular attachment and poor secretion, necessitating cell-wall extraction and extensive purification of the enzyme with poor yield (e.g. Ishikawa et al. (2005) Journal of Bioscience and Bioengineering, 4: 331-339).

To alleviate the problems with production of these GOS-producing enzymes, the gene sequence of a β-hexosyltransferase (BHT) from Sporobolomyces singularis was determined (Ishikawa et al (2005)). In an attempt to over-express BHT, the gene was cloned in different Escherichia coli strains but all enzyme was found to be insoluble, even when it was expressed as fusion protein to enhance solubility (Dagher et al (2013) Applied and Environmental Microbiology, 79: 1241-1249). Expression of the same gene in Pichia pastoris in frame with an α-mating factor signal sequence (αMF) did, however, result in the secretion of a small amount of BHT. The highest secretion of 9.8 mg BHT per liter culture was found using a construct where a potential secretion signal at position 1-22 of the BHT sequence was deleted and the αMF signal sequence was used instead, and a HIS tag was attached to the C-terminal end of the BHT sequence (WO2014/089558 A1). It was concluded in WO2014/089558 that “neither αMF nor the BHT leader secretion signal could fully complete the secretion of rBHT-HIS which may be related to the presence of a transmembrane region predicted between amino acids 177-199”.

The inventors of the present applications have been able to produce the beta-hexosyltransferase from Sporobolomyces singularis in sufficient amounts using K. lactis and A. niger as a production host. Both hosts produce sufficient quantities of a hexosyltransferase from Sporobolomyces singularisis. Preferably, the used hexosyltransferase from Sporobolomyces singularisis a truncation variant of the wildtype sequence. An example of a suitable variant is described herein within the experimental part.

Preferably, the used hexosyltransferase from Sporobolomyces singularis (produced in A. niger or K. lactis) has at least 80% sequence identity to SEQ ID NO: 1 or 2. For the purpose of this invention, it is defined herein that in order to determine the percentage of sequence identity of two amino acid sequences or of two nucleic acid sequences, the sequences are aligned for optimal comparison purposes. In order to optimize the alignment between the two sequences gaps may be introduced in any of the two sequences that are compared. Such alignment can be carried out over the full length of the sequences being compared. In case specific positions are described these positions should also be used in a comparison. The sequence identity is the percentage of identical matches between the two sequences over the reported aligned region.

A comparison of sequences and determination of percentage of sequence identity between two sequences can be accomplished using a mathematical algorithm. The skilled person will be aware of the fact that several different computer programs are available to align two sequences and determine the identity between two sequences (Kruskal, J. B. (1983) An overview of sequence comparison In D. Sankoff and J. B. Kruskal, (ed.), Time warps, string edits and macromolecules: the theory and practice of sequence comparison, pp. 1-44 Addison Wesley). The percentage of sequence identity between two amino acid sequences or between two nucleotide sequences may be determined using the Needleman and Wunsch algorithm for the alignment of two sequences. (Needleman, S. B. and Wunsch, C. D. (1970) J. Mol. Biol. 48, 443-453). Both amino acid sequences and nucleotide sequences can be aligned by the algorithm. The Needleman-Wunsch algorithm has been implemented in the computer program NEEDLE. For the purpose of this invention the NEEDLE program from the EMBOSS package was used (version 2.8.0 or higher, EMBOSS: The European Molecular Biology Open Software Suite (2000) Rice, P. Longden, I. and Bleasby, A. Trends in Genetics 16, (6) pp276-277, http://emboss.bioinformatics.nl/). For protein sequences EBLOSUM62 is used for the substitution matrix. For nucleotide sequence, EDNAFULL is used. The optional parameters used are a gap-open penalty of 10 and a gap extension penalty of 0.5. The skilled person will appreciate that all these different parameters will yield slightly different results but that the overall percentage identity of two sequences is not significantly altered when using different algorithms.

After alignment by the program NEEDLE as described above the percentage of sequence identity between a query sequence and a sequence of the invention is calculated as follows: Number of corresponding positions in the alignment showing an identical amino acid or identical nucleotide in both sequences divided by the total length of the alignment after subtraction of the total number of gaps in the alignment. The identity defined as herein can be obtained from NEEDLE by using the NOBRIEF option and is labeled in the output of the program as “longest-identity”.

Alternatively, the used hexosyltransferase from Sporobolomyces singularis (produced in A. niger or K. lactis) has at least 85%, 90% or 95, 96, 97, 98 or 99% sequence identity to SEQ ID NO: 1 or 2.

Examples of suitable milk-based substrates have been provided above and may be skim milk, semi-skimmed milk, full fat milk, pasteurized milk, UHT milk, milk having an extended shelf life, condensed milk, reconstituted (skim) milk prepared from powder, butter milk, milk protein concentrate (MPC), or whey such as sweet whey or acid whey, whey protein concentrate (WPC), or whey permeate.

In one of the aspects of the invention, the level of lactose has not been reduced in the milk-based substrate, for example the milk-based substrate has not been subjected to lactose reducing filtration techniques. In yet another aspect, the level of lactose in the milk-based has been reduced before incubation with the first and second enzyme.

A method of the invention may result in increased sweetness which is not appreciated by all people. Increased sweetness can be reduced by reducing part of the lactose present in a milk-based substrate by using well known techniques. I.e. a method of the invention may include a step to reducing the level of lactose in the milk-based substrate before treating said substrate with a first and second enzyme as described above.

The level of lactose in a milk-based substrate may be increased, for example by concentration, before treating the milk-based substrate with a first and second enzyme as described above. A typical production process for producing lactose reduced (preferably lactose free) dairy (preferably milk) powder comprises a concentration step. The evaporation step as shown in FIG. 1 is a concentration step and the enzymes used in a method of the invention can be added during and/or after evaporation. Preferably, the amount of lactose in a milk-based substrate is within the range of 4 to 10 (w/v) % lactose, preferably 4 to 8 (w/v) % and even more preferably 4 to 6 (w/v) % or 4.5 to 5.5 (w/v) % lactose.

The first and second enzyme which are used in any of the above described methods can be added simultaneously or sequentially. The inventors of the present invention noted that the best results are obtained by using the first and second enzyme sequentially wherein the enzyme having transgalactosylase activity is added first. Thus, first an amount of GOS is produced from lactose and the remaining lactose is hydrolysed to glucose and galactose by the second enzyme having lactase (hydrolytic) activity.

The invention thus provides a method for producing a lactose reduced (preferably lactose free) dairy powder (preferably milk powder) comprising
(i) treating a lactose comprising milk-based substrate with a first enzyme having transgalactosylase activity and a second enzyme having lactase activity to obtain a lactose reduced (preferably lactose free) and galacto-oligosaccharides (GOS) comprising milk-based product
(ii) preparing lactose reduced (preferably lactose free) dairy powder (preferably milk powder) from said lactose reduced (preferably lactose free) and GOS comprising milk-based product,

wherein the milk-based substrate is first treated with the enzyme having transgalactosylase activity and subsequently with the enzyme having lactase activity, preferably said method does not comprise an enzyme inactivation step between the treatment with the enzyme having transgalactosylase activity and the treatment with the enzyme having lactase activity.

The skilled person is aware that some enzymes having lactase activity are capable of reducing GOS levels, especially when incubated for extended periods. The skilled person will take precautions to avoid (excessive) reduction of GOS by the enzyme having lactase activity.

The inventors of the present invention noted that very good results are obtained by using the first and second enzyme simultaneously. Thus, production of GOS from lactose by the first enzyme having transgalactosylase activity and lactose hydrolysis to glucose and galactose by the second enzyme having lactase (hydrolytic) activity are taking place at the same time.

The invention thus provides a method for producing a lactose reduced (preferably lactose free) dairy powder (preferably milk powder) comprising
(i) treating a lactose comprising milk-based substrate with a first enzyme having transgalactosylase activity and a second enzyme having lactase activity to obtain a lactose reduced (preferably lactose free) and galacto-oligosaccharides (GOS) comprising milk-based product
(ii) preparing lactose reduced (preferably lactose free) dairy powder (preferably milk powder) from said lactose reduced (preferably lactose free) and GOS comprising milk-based product,

wherein step (i) is treating a lactose comprising milk-based substrate simultaneously with a first enzyme having transgalactosylase activity and a second enzyme having lactase activity to obtain a lactose reduced and galacto-oligosaccharides (GOS) comprising milk-based product.

Examples of dairy powder have been described above: a dairy powder are milk powder or whey powder. Examples of milk powder are skim milk powder, semi-skimmed milk powder, full fat milk powder, butter milk powder, MPC powder, and examples of whey powder are sweet whey powder or acid whey powder, WPC powder, and whey permeate powder. Preferably, the dairy powder is milk powder or whey powder. More preferably, the dairy powder is milk powder and most preferably, the dairy powder is lactose free milk powder.

The invention also provides a dairy powder, preferably a dairy powder obtainable by a method as described above.

A preferred dairy powder is a lactose free dairy powder comprising at least 4% (w/w) GOS.

The lactose free powder comprises at least 4% (w/w) GOS and <0.1 (w/w) % lactose. The invention thus provides a dairy powder (preferably milk powder) comprising at least 4% (w/w) GOS and <0.1% (w/w) lactose.

The amount of GOS in a dairy powder can be determined by subtracting the lactose, glucose and galactose concentration in a dairy powder, from the total amount of monosugars (glucose and galactose) after complete hydrolysis of the dairy powder with an acid lactase (based on AOAC official method 2001.02; described in the experimental part herein in more detail).

In yet another embodiment, the invention provides lactose free milk comprising at least 10% (w/w) GOS based on total carbohydrates and having a glucose/galactose ratio of >1.2 or the invention provides a milk comprising <0.01% lactose, at least 10% (w/w) GOS based on total carbohydrates and having a glucose/galactose ratio of >1.2.

Preferably, the (lactose-free) milk is UHT milk. Prior art lactose reduced UHT milk comprises considerable amounts of mono-sugars and the presence of these mono-sugars results in a Maillard reaction which subsequently results in browning of the UHT milk (especially when stored for longer periods). Especially galactose is reactive in the Maillard reaction, and hence lactose-free UHT milk with an increase in both GOS content and an increase in the ratio glucose/galactose will have a reduced chance in browning during storage. UHT milk comprising <0.01% lactose, at least 10% (w/w) GOS based on total carbohydrates and having a glucose/galactose ratio of >1.2 does not comprise considerable amounts of galactose and hence reduced or no Maillard/browning.

The inventors of the present invention show herein that the presence of GOS (preferably in situ produced GOS) in a lactose reduced milk based substrate has advantages when preparing a powder from the lactose reduced milk-based substrate. The advantages can be described as an advantage in the efficiency of the drying process and/or as advantages of the final product (i.e. dairy (preferably milk) powder) Examples of these advantages are (when compared to a lactose reduced or a lactose free milk based substrate which does not or hardly not comprise GOS): improving (increasing) the drying capacity of (spray) dryer, improving (increasing) the drying speed of the dryer, improving (reducing) the production time of the drying process, improving (reducing) the hygroscopic behaviour of lactose reduced (preferably lactose free) dairy powder, improving (reducing) the stickiness of lactose reduced (preferably lactose free) dairy powder and/or improving (reducing) the brown color formation of lactose reduced milk powder upon storage

Hence, in yet another embodiment, the invention therefore provides

I Use of GOS for improving drying of a lactose reduced milk-based substrate. Preferably, the GOS is in situ produced GOS.

Use of GOS for improving drying of a lactose free milk-based substrate. Preferably, the GOS is in situ produced GOS.

Use of GOS for increasing the drying capacity for lactose reduced milk-based substrate. Preferably, the GOS is in situ produced GOS.

Use of GOS for increasing the drying capacity for lactose free milk-based substrate. Preferably, the GOS is in situ produced GOS.

Use of GOS for increasing the drying speed of lactose reduced milk-based substrate. Preferably, the GOS is in situ produced GOS.

Use of GOS for increasing the drying speed of lactose free milk-based substrate. Preferably, the GOS is in situ produced GOS.

Use of GOS for reducing the production time of a lactose reduced dairy powder from a lactose reduced milk-based substrate. Preferably, the GOS is in situ produced GOS.

Use of GOS for reducing the production time of a lactose free dairy powder from a lactose free milk-based substrate. Preferably, the GOS is in situ produced GOS.

Use of GOS for reducing the hygroscopic behaviour of a lactase reduced dairy powder. Preferably, the GOS is in situ produced GOS.

Use of GOS for reducing the hygroscopic behaviour of a lactase free dairy powder. Preferably, the GOS is in situ produced GOS.

Use of GOS for reducing the stickiness of a lactase reduced dairy powder. Preferably, the GOS is in situ produced GOS.

Use of GOS for reducing the stickiness of a lactase free dairy powder. Preferably, the GOS is in situ produced GOS.

Use of GOS for reducing brown color formation of lactose reduced milk powder. Preferably reduce brown color formation upon storage. Preferably, the GOS is in situ produced GOS.

Use of GOS for reducing brown color formation of lactose free milk powder. Preferably reduce brown color formation upon storage. Preferably, the GOS is in situ produced GOS.

Use of an enzyme having transgalactosylase activity and an enzyme having lactase activity for improving the drying of a lactose reduced milk-based substrate.

Use of an enzyme having transgalactosylase activity and an enzyme having lactase activity for improving the drying of a lactose reduced milk-based substrate.

Use of an enzyme having transgalactosylase activity and an enzyme having lactase activity for improving a characteristic of lactose free milk powder.

The features and definitions which are given above in the context of the method claims, equally apply to the use claims.

The invention also provides additional embodiments:
1. An isolated and/or recombinant polypeptide having transgalactosylase activity and having at least 95% sequence identity with SEQ ID NO: 2.
2. A polypeptide according to embodiment 1 having at least 96%, 97%, 98% or 99% sequence identity with SEQ ID NO: 2.
3. A polypeptide according to embodiment 1 or 2 which consists of SEQ ID NO: 2.
4. A method for producing a dairy product comprising adding an effective amount of a polypeptide according to any one of embodiments 1 to 3 to a milk substrate and allowing the polypeptide to exert its enzymatic activity.
5. A composition comprising a polypeptide according to any one of embodiments 1 to 3 and at least one component selected from salt, preservative, polyol or metal ions.
6. A dairy product obtainable by the method of embodiment 4.
7. A dairy product comprising a polypeptide according to any one of embodiments 1 to 3.
8. Use of a polypeptide according to any one of embodiments 1 to 3 for producing GOS, preferably in situ GOS.

The present invention is further illustrated by the following non-limiting Examples.

### Materials and Methods

### Enzymes Used in This Study

1. Maxilact LGi5000 (DSM Food Specialties): beta-galactosidase from Kluyveromyces lactis. The used Maxilact LGi5000 sample had an activity of 5000 NLU/ml
2. Biolacta FN5 (Amano): beta-galactosidase from Bacillus circulans. The enzyme was glycated prior to use by performing the protocol as described in WO2018/210820. For this, Biolacta FN5 enzyme powder was dissolved at 10% (w/v) in 60% glucose, and incubated at 50° C. for 66 hr. The used Biolacta FN5 sample had an activity of 225 NLU/mg (before glycation).
3. BHT: β-hexosyltransferase, a transgalactosidase originating from Sporobolomyces singularis. The used BHT samples had an activity of 124 WBDG/ml. Procedures to produce an experimental sample of this enzyme have been described in the past (Dagher et al. (2013) Appl. Environm. Microbiol. 79 (4) 1241-1249). For this the gene encoding the BHT enzyme was cloned behind a strong promoter in a suitable production host, like Kluyveromyces lactis or Aspergillus niger, using standard technology. After fermentation of the selected recombinant expression strain, the secreted enzyme was isolated from the cleared fermentation broth and concentrated using ultrafiltration. More details on the production of a truncated BHT enzyme production are described below.

### SMP Used in This Study

Nilac skimmed milk powder (SMP), NIZO, The Netherlands was used. Typical composition is 35% protein, 52% lactose, 1% fat, 8% ash and 4% moisture.

### Production of a Truncated BHT Enzyme

### Design of Truncated BHT

In order to predict where the wildtype full length protein sequence can be truncated the location of a linker domain was determined. The linker domain separates the putative N-terminal cell associated domain (CAD) from the catalytic domain in the enzyme. Truncation anywhere in this linker region likely results in functional soluble enzyme, as the functional domain don't lose its structural integrity. Cutting in the middle of a domain likely results in problems with the structural architecture of the enzyme, which can lead to protease sensitivity, misfolding, aggregation or other problems that prevent formation of a stable soluble enzyme product. To determine the linker region in SEQ ID NO:1, an online prediction server like IUPRED (http://iupred.enzim.hu/) was used (settings: prediction type: structured regions, generate plot). Running this tool for SEQ ID NO:1, the output marks the N-terminal linker region running from position 38 until 55 in SEQ ID NO:1 . To separate the CAD from the catalytic domain, a deletion is most preferably made of the CAD and ending somewhere in the linker domain.

### References

The Pairwise Energy Content Estimated from Amino Acid Composition Discriminates between Folded and Intrinsically Unstructured Proteins—Zsuzsanna Dosztányi, Veronika Csizmók, Péter Tompa and István Simon
J. Mol. Biol. (2005) 347, 827-839.
IUPred: web server for the prediction of intrinsically unstructured regions of proteins based on estimated energy content—Zsuzsanna Dosztányi, Veronika Csizmók, Péter Tompa and István Simon
Bioinformatics (2005) 21, 3433-3434.

### Cloning of Truncated BHT

Two constructs with different truncations in BHT of Sporobolomyces singularis were designed.
BHT #132: Wild-type construct without any deletion (SEQ ID NO:1)
BHT #134: Deletion starts at position 18 up to position 54 (SEQ ID NO:2)

Codon-adapted DNA sequences for expression of the proteins in Aspergillus niger were designed containing additional restriction sites for subcloning in an Aspergillus expression vector. Codon adaptation was performed as described in WO2008/000632. The codon optimized DNA sequences for expression of these genes encoding the BHT#132 and BHT#134 protein of SEQ ID NO: 1 and SEQ ID NO: 2 in A. niger are shown in SEQ ID NO: 3 and SEQ ID NO: 4, respectively.
The translational initiation sequence of the glucoamylase glaA promoter was modified into 5′-CACCGTCAAA ATG-3′ (SEQ ID NO:5) and an optimal translational termination sequence 5′-TAAA-3′ was used in the generation of the expression constructs (as also detailed in WO2006/077258). The DNA fragments (SEQ ID NO: 6 and SEQ ID NO: 7), containing a.o. part of the glucoamylase promoter and the BHT #132 and BHT #134 encoding genes, was synthesized completely, purified and digested with EcoRI and PacI.

The pGBTOP-16 vector was linearized by EcoRI/PacI digestion and the linearized vector fragment was subsequently purified by gel-extraction. The DNA fragments were cloned into the pGBTOP-16 vector and the resulting vectors were named pGBTOPBHT #132 and pGBTOPBHT #134. Subsequently, A. niger GBA 306 was transformed with these vectors in a co-transformation protocol with pGBAAS-4, with strain and methods as described in WO 2011/009700 and references therein, and selected on acetamide containing media and colony purified according to standard procedures. Transformation and selection was performed as described in WO 98/46772. Strains containing the

BHT#132 and BHT#134 genes were selected via PCR with primers amplifying the introduced BHT#132 and BHT#134 gene to verify presence of the BHT expression cassettes. Two transformants from each variant expressing the BHT#132 or BHT#134 genes were selected, and further replica-plated to obtain a single strain inoculum.

Fermentation of A. niger Strains in Microtiter Plate
Fresh A. niger spores from the strains generated above 2 were prepared and used for generating sample material by cultivation of the strain in 24 deep well plates (Axygen, Union City, USA) containing 3 ml fermentation medium 2 (15% w/v maltose, 6% w/v bacto-soytone, 1.5% w/v (NH4)2SO4, 0.1% w/v NaH2PO4.H20, 0.1% w/v MgSO4.7H2O, 0.1% w/v L-arginine, 8% w/v Tween-80, 2% w/v Basildon, 2% w/v MES, pH 6.2). The 24 deep well plates were covered with a Breathseal (Greiner bio-one, Frickenhausen, Germany) and a lid. After 6 days of growth at 34° C., 550 rpm and 80% humidity in a Microton incubator shaker (Infors AG, Bottmingen, Switzerland) 1.5 mL samples were taken, the mycelium was separated from the supernatant by centrifugation for 30 min at 4000 g and the supernatants were stored at −20° C. until further analyses.

Small amounts of the supernatants of the different cultures were analyzed on SDS-PAGE and stained using Coomassie Brilliant Blue. All transformants showed protein bands of the expected size. For example, both transformants harboring construct BHT #134 showed a clear secretion of a protein with apparent molecular weight of ˜70 kDa. This size is slightly smaller than the size estimated for the wild-type BHT isolated from S. singularis biomass (Ishikawa et al, 2005), but much smaller than the 110 kDa recombinant BHT expressed in Pichia pastoris (Dagher et al, 2013). This suggests that expression of BHT #134 in Aspergillus niger results in production of an enzyme that is more similar to the native Sporobolomyces BHT.

Expression of BHT#134 in shake flask

The A. niger transformant strains were used for generation of sample material by cultivation of the strains in shake flask cultures. A useful method for cultivation of A. niger strains and separation of the mycelium from the culture broth is described in WO 98/46772. Cultivation was in CSM-MES (150 g maltose, 60 g Soytone (Difco), 15 g (NH4)2SO4, 1 g NaH2PO4.H2O, 1 g MgSO4.7H20, 1 g L-arginine, 80 mg Tween-80, 20 mg Basildon, 20 g MES pH6.2 per liter medium). The shake flasks were incubated at 280 rpm and 30° C. Samples were taken from duplicate cultivations on day 6 of the fermentation, centrifuged for 10 min at 5000 rpm in a Hereaus labofuge RF and supernatants were stored at −20° C. until further analyses.

In order to obtain an estimate of the productivity of the BHT#134 transformants, all fermentation supernatants were analyzed using Bradford total protein analysis according to manufacturer's instructions (Sigma—B6916), and as also used in WO2014089558. Total protein concentration in the samples having no or hardly any additional protein expressed, were averaged and compared to the average of the samples of the BHT#134 transformants. Productivity is expressed as the difference between BHT#134 sample and the control. Results are depicted in Table 1.

TABLE 1
Productivity of BHT#134. Control is a similar
cultivation of non-transformed Aspergillus niger
CBS513.88. WO2014089558 indicates maximum prior art
productivity of wild-type BHT in Pichia pastoris.

| | | | Protein | Produc- |
|---|---|---|---|---|
| | Cultivation | Cultivation | concentration | tivity |
| | method | time (days) | (mg/l) | (mg/l) |
| Controls | MTP | 6 | 240 ± 48 | — |
| BHT#134 | MTP | 6 | 550 ± 15 | 310 |
| Control | Shake flask | 6 | 210 | — |
| BHT#134 | Shake flask | 6 | 620 | 410 |
| WO2014089558 | Shake flask | 6 | | 9.8 |

This productivity in MTP and shakeflask is much higher than the secretion of any basidiomycetous BHT described in the prior art. To our knowledge the best secretion of such enzymes was obtained with the Sporobolomyces singularis BHT in which the regular signal sequence was replaced by an αMF signal sequence (WO2014089558). Secretion of this construct in Pichia pastoris led to a productivity of 9.8 mg BHT/I in shake flask, more than a factor 50 lower than BHT #134 secretion.

### Activity Determination Maxilact LGi5000 and Biolacta FN5

Beta-galactosidase activity in Maxilact LGi5000 and Biolacta FN5 was determined using the neutral lactase activity (NLU) assay essentially performed as described in the Food Chemical Codex (FCC 6) page 1124-1126.

### Activity Determination BHT

The activity of BHT was determined using an assay which is essentially similar to the assay described (Workman and Day (1982) Appl. Environm. Microbiol. 44(6) 1289-1295). Assay conditions were slightly adapted and performed at 37° C. in sodium acetate buffer pH 4.40 at a p-nitrophenyl-p-D-glucopyranoside (PNPG) concentration of 4.7 mM. After termination of the reaction the pNP released was measured at 405 nm, and the enzyme activity was expressed as nanomole of p-nitrophenol released per second (BHTU).

### Determination of the Lactose, Glucose, Galactose and GOS Content

Lactose, glucose and galactose content in milk-based samples were determined using a HPAEC-PAD method essentially as described by the AOAC (“AOAC official method 2001. 02 trans-Galactooligosaccharides (TGOS),” in Selected Food Products, AOAC International, Gaithersburg, Md, USA, 2005).

When the milk-based substrate and the milk-based product as described within this patent application are both available (as is the case in Example 3), the lactose, glucose and galactose concentration can be determined in both the milk-based substrate and in the milk-based product. The total GOS concentration in the milk-based product can be calculated by subtracting the lactose, glucose and galactose content in the milk-based product from the one in the milk-based substrate with the formula (1):

[GOS]_{product}=([lac]+[glu]+[gal])_{substrate}−([lac]+[glu]+[gla])_{product.} (1)

[lac] =concentration lactose in g/L

[glu] =concentration glucose in g/L

[gal] =concentration galactose in g/L

[GOS] =concentration galacto-oligosaccharides in g/L

When the GOS content has to be determined in a sample of a milk-based product or milk-based powder without having the milk-based substrate available, the total galacto-oligosaccharide (GOS) content of the milk-based samples can be determined with a method similar to the standard AOAC method (“AOAC official method 2001. 02 trans-Galactooligosaccharides (TGOS),” in Selected Food Products, AOAC International, Gaithersburg, Md, USA, 2005). With the described HPAEC-PAD method the galactose, glucose and lactose concentration in the samples can be determined, before (initial) and after (final) hydrolysis of the samples with an acid lactase. The GOS content in the initial sample can be calculated by subtracting the initial measured amount of sugars from the final measured amount of sugars with the following formula (2):

[GOS]ᵢₙᵢₜᵢₐₗ=([lac]+[glu]+[gal])_{final}−([lac]+[glu]+[gla])ᵢₙᵢₜᵢₐₗ (2)

[lac] =concentration lactose in g/L or g/kg

[glu] =concentration glucose in g/L or g/kg

[gal] =concentration galactose in g/L or g/kg

[GOS] =concentration galacto-oligosaccharides in g/L or g/kg
Total carbohydrates in a milk-based substrate, a milk-based product or a milk powder is defined as the sum of the concentrations of glucose, galactose, lactose and GOS.

### Determination of Glass Transition Temperature (Tg) by Differential Scanning Calorimetry (DSC)

Milk powder samples of 2.5 mg mass are weighed with a precision balance and encapsulated in (crimped) aluminum pans of known mass. An identical empty pan is used as a reference. Nitrogen is purged at a rate of 50 ml min−1. Heating-Cooling-Heating-Cooling-Heating cycles at 10K/min, in the range −80 until 120° C., are applied in a DSC apparatus (METLLER DSC 3+). A residence time of 0.5 minutes was selected at the maximum temperature for allowing moisture vaporization, simultaneous with DSC signal equilibration. Three heating-cooling cycles were performed. The first heating cycle was mainly used for evaporation of water while the 2^{nd} and 3^{rd} cycle were performed to measure Tg and establish reproducibility of the measurement.

### EXAMPLE 1

### Skim-Milk Treated With BHT and Maxilact

A 10% (w/v) skim milk powder (SMP) solution was produced by dispersing 100 gram SMP in 900 ml tap water (40° C.) under agitation (250 rpm) during 30 min. After resting for 30 minutes at room temperature the dispersion was agitated during 24 h at 150 rpm 4° C. 5 ml samples of this milk was used for treatment with BHT for 24 hr at 8° C. BHT dosage was 1, 2, 5, or 10 BHTU/ml.

Additionally 1 or 2.5 NLU/ml Maxilact LGi5000 was added at the same time with BHT, or 5 or 10 NLU/ml Maxilact LGi5000 was added after 18 hr (so incubation time with Maxilact was limited to 6 hr). Control samples were SMP solution where no enzymes were added (control A), incubation with only Maxilact at 5 NLU/ml (control C) and incubation with only BHT at 5 BHTU/ml for 24 h (control D). All reactions were stopped after incubation by heating for 10 minutes at 95° C.

Samples were split in 1 ml for sugar analysis and 4 ml portions were stored frozen for later analysis

TABLE 2
Skim milk samples treated with BHT and Maxilact. Dosage of
the enzymes and incubation time with added BHT (incubation
time 1) or added Maxilact (incubation time 2) is indicated.

| | | | Incubation | | | Incubation |
|---|---|---|---|---|---|---|
| | | Enzyme 1 | time 1 | | Enzyme 2 | time 2 |
| Sample | Enzyme 1 | BHTU/ml | (hours) | Enzyme 2 | NLU/ml | (hours) |
| 1 | BHT | 1 | 24 | Maxilact | 1 | 24 |
| 2 | BHT | 2 | 24 | Maxilact | 1 | 24 |
| 3 | BHT | 5 | 24 | Maxilact | 1 | 24 |
| 4 | BHT | 10 | 24 | Maxilact | 1 | 24 |
| 5 | BHT | 1 | 24 | Maxilact | 2.5 | 24 |
| 6 | BHT | 2 | 24 | Maxilact | 2.5 | 24 |
| 7 | BHT | 5 | 24 | Maxilact | 2.5 | 24 |
| 8 | BHT | 10 | 24 | Maxilact | 2.5 | 24 |
| 9 | BHT | 1 | 24 | Maxilact | 5 | 6 |
| 10 | BHT | 2 | 24 | Maxilact | 5 | 6 |
| 11 | BHT | 5 | 24 | Maxilact | 5 | 6 |
| 12 | BHT | 10 | 24 | Maxilact | 5 | 6 |
| 13 | BHT | 1 | 24 | Maxilact | 10 | 6 |
| 14 | BHT | 2 | 24 | Maxilact | 10 | 6 |
| 15 | BHT | 5 | 24 | Maxilact | 10 | 6 |
| 16 | BHT | 10 | 24 | Maxilact | 10 | 6 |
| Control A | | 0 | | | 0 | 24 |
| Control C | | 0 | | Maxilact | 5 | 24 |
| Control D | BHT | 5 | 24 | | 0 | |

### EXAMPLE 2

### Skim Milk Treated With Glycated Biolacta and Maxilact

A 10% (w/v) skim milk powder (SMP) solution was produced by dispersing 100 gram SMP in 900 ml tap water (40° C.) under agitation (250 rpm) during 30 min. After resting for 30 minutes at room temperature the dispersion was agitated during 24 h at 150 rpm 4° C. 5 ml samples of this milk was used for treatment with glycated Biolacta FN5 for 24 hr at 8° C. Enzyme dosage of the glycated Biolacta FN5 preparation was 1, 2, 5 or 10 mg/ml skim milk. Additionally 1 and 2.5 NLU/ml Maxilact LGi5000 was added at the same time with glycated Biolacta, or 5 and 10 NLU/ml Maxilact LGi5000 was added after 18 hr (so incubation time with Maxilact was limited to 6 hr).

Control samples were SMP solution where no enzymes were added (control A1), incubation with only Maxilact at 5 NLU/ml (control C1) and incubation with only Biolacta at 10 mg/ml for 24 h (control D). All reactions were stopped after incubation for 24 h by heating for 10 minutes at 95° C. Samples were split in 1 ml for sugar analysis and 4 ml portions were stored frozen for later analysis.

TABLE 3
Skim milk samples treated with glycated Biolacta and Maxilact. Dosage
of the enzymes and incubation time with added glycated Biolacta (incubation
time 1) or added Maxilact (incubation time 2) is indicated.

| | | | Incubation | | | Incubation |
|---|---|---|---|---|---|---|
| | | Enzyme 1 | time 1 | | Enzyme 2 | time 2 |
| Sample | Enzyme 1 | mg/ml | (hours) | Enzyme2 | NLU/ml | (hours) |
| 1A | BioLacta | 1 | 24 | Maxilact | 1 | 24 |
| 2A | BioLacta | 2 | 24 | Maxilact | 1 | 24 |
| 3A | BioLacta | 5 | 24 | Maxilact | 1 | 24 |
| 4A | BioLacta | 10 | 24 | Maxilact | 1 | 24 |
| 5A | BioLacta | 1 | 24 | Maxilact | 2.5 | 24 |
| 6A | BioLacta | 2 | 24 | Maxilact | 2.5 | 24 |
| 7A | BioLacta | 5 | 24 | Maxilact | 2.5 | 24 |
| 8A | BioLacta | 10 | 24 | Maxilact | 2.5 | 24 |
| 9A | BioLacta | 1 | 18 | Maxilact | 5 | 6 |
| 10A | BioLacta | 2 | 18 | Maxilact | 5 | 6 |
| 11A | BioLacta | 5 | 18 | Maxilact | 5 | 6 |
| 12A | BioLacta | 10 | 18 | Maxilact | 5 | 6 |
| 13A | BioLacta | 1 | 18 | Maxilact | 10 | 6 |
| 14A | BioLacta | 2 | 18 | Maxilact | 10 | 6 |
| 15A | BioLacta | 5 | 18 | Maxilact | 10 | 6 |
| 16A | BioLacta | 10 | 18 | Maxilact | 10 | 6 |
| Control A1 | | 0 | | | 0 | 24 |
| Control C1 | | 0 | | Maxilact | 5 | 24 |
| Control D1 | BioLacta | 10 | 24 | | 0 | |

### EXAMPLE 3

### Sugar Analysis

The sugar composition (glucose, galactose and lactose) of all samples generated in Examples 1 and 2 was analysed as described in the Materials and Methods section. The GOS content of each sample was calculated from the content of the measured sugars, as described in this section. The glucose content measured in the samples of Example 2 was corrected for the amount of glucose that was added in the sample via the glycated Biolacta sample. E.g., in control D1 10 mg/ml glycated Biolacta with 60% glucose is added, so 6 g/L glucose has been subtracted from the measurement.

TABLE 4
Lactose, glucose, galactose and GOS content
of samples generated in Example 1

| | | | | Total | |
|---|---|---|---|---|---|
| | Lactose | Glucose | Galactose | GOS | Ratio |
| Sample | (g/L) | (g/L) | (g/L) | (g/L) | glu/gal |
| 1 | 5.5 | 22.8 | 17.8 | 9.5 | 1.28 |
| 2 | 3.6 | 23.2 | 17.8 | 11.0 | 1.30 |
| 3 | 2.1 | 23.4 | 17.5 | 12.6 | 1.34 |
| 4 | <0.1 | 22.3 | 16.2 | 17.1 | 1.38 |
| 5 | <0.1 | 25.9 | 22.2 | 7.5 | 1.17 |
| 6 | <0.1 | 25.7 | 22.0 | 7.9 | 1.17 |
| 7 | <0.1 | 25.9 | 22.3 | 7.4 | 1.16 |
| 8 | <0.1 | 24.0 | 20.6 | 11.0 | 1.17 |
| 9 | 1.9 | 24.2 | 19.3 | 10.2 | 1.25 |
| 10 | 1.4 | 23.6 | 18.2 | 12.4 | 1.30 |
| 11 | 1.1 | 21.8 | 16.0 | 16.7 | 1.36 |
| 12 | 1.1 | 19.3 | 12.5 | 22.7 | 1.54 |
| 13 | <0.1 | 26.5 | 21.7 | 7.4 | 1.22 |
| 14 | <0.1 | 24.4 | 20.0 | 11.2 | 1.22 |
| 15 | <0.1 | 23.2 | 18.4 | 14.0 | 1.26 |
| 16 | <0.1 | 21.0 | 16.2 | 18.4 | 1.30 |
| Control A | 55.6 | <0.1 | <0.1 | <0.1 | n.a. |
| Control C | <0.1 | 27.8 | 25.3 | 2.5 | 1.10 |
| Control D | 32.4 | 5.3 | <0.1 | 17.9 | >53.00 |

TABLE 5
Lactose, glucose, galactose and GOS content
of samples generated in Example 2

| | | Glucose | | Total | |
|---|---|---|---|---|---|
| | Lactose | corrected | Galactose | GOS | Ratio |
| Sample | (g/L) | (g/L) | (g/L) | (g/L) | glu/gal |
| 1A | 4.1 | 22.0 | 17.0 | 6.3 | 1.29 |
| 2A | 4.0 | 21.6 | 16.3 | 7.5 | 1.33 |
| 3A | 4.2 | 22.5 | 16.4 | 6.3 | 1.37 |
| 4A | 3.5 | 20.4 | 15.7 | 9.8 | 1.30 |
| 5A | <0.1 | 26.1 | 22.2 | 1.1 | 1.18 |
| 6A | <0.1 | 24.8 | 21.1 | 3.5 | 1.18 |
| 7A | <0.1 | 23.9 | 20.7 | 4.8 | 1.15 |
| 8A | <0.1 | 23.2 | 21.0 | 5.2 | 1.10 |
| 9A | <0.1 | 23.1 | 17.8 | 8.5 | 1.30 |
| 10A | <0.1 | 23.6 | 17.6 | 8.2 | 1.34 |
| 11A | 1.3 | 21.6 | 15.2 | 11.3 | 1.42 |
| 12A | 2.2 | 20.4 | 14.7 | 12.1 | 1.39 |
| 13A | <0.1 | 24.3 | 19.4 | 5.7 | 1.25 |
| 14A | <0.1 | 23.5 | 18.4 | 7.5 | 1.28 |
| 15A | <0.1 | 23.0 | 17.5 | 8.9 | 1.31 |
| 16A | <0.1 | 21.5 | 16.7 | 11.2 | 1.29 |
| Control A1 | 49.4 | <0.1 | <0.1 | <0.1 | n.a. |
| Control C1 | <0.1 | 25.5 | 21.9 | 2.0 | 1.16 |
| Control D1 | 12.4 | 14.9 | 8.6 | 13.5 | 1.73 |

Full conversion of all lactose and GOS in the analytical method was checked by calculating the mass balance in each sample and comparing to the initial lactose content of the untreated skim milk, and was found to be satisfactory.
The results show that it is possible to generate skim milk samples with low lactase and an appreciable amount (>10% (w/w) of total carbohydrates) of GOS. Best results are obtained with the combination of BHT and Maxilact (samples #4-8 and #13-16), but also the combination of glycated Biolacta and Maxilact gives a satisfactory result (samples #13A-16A).

### EXAMPLE 4

### Determination of Tg on Milk Powder Samples

Samples Control A, Control C, sample 4 and sample 16 (all described in Example 1) were freeze dried and the glass transition temperature (Tg) of the powders was determined using differential scanning calorimetry (DSC) as described in the Materials and Methods section. All DSC first heating curves yielded a broad endothermic peak very likely reflecting water vaporization. Subsequent DSC heating ramps (e.g. 2^{nd} respectively 3^{rd} heating) were conducted to a maximum temperature of 120 degrees C. and yielded reproducible discontinuities in the heat capacity which can be interpreted as glass transition phenomena; reproducibility between the 2^{nd} and the 3^{rd} heating ramp shows that on the one hand that water removal was successful, and also that eventual thermal induced degradation did not occur during the experiment. Tg was calculated from the onset temperature of the discontinuities in the heat capacity of the 2^{nd} and 3^{rd} temperature cycle, and is depicted in Table 6. The onset temperature is defined as the intersection point of the baseline before transition and the inflectional tangent.

TABLE 6
Tg of freeze dried skim milk samples treated
with BHT and Maxilact, compared to controls.

Tmax 120 degrees C.

| | 2nd Heating | 3rd Heating |
|---|---|---|
| Sample | Tg, C | Tg, C |
| Control A | | 119.1 |
| Control C | 47.3 | 47.6 |
| Sample 4 | 70.5 | 70 |
| Sample 4 Duplicate | 70.7 | 70.5 |
| Sample 16 | 63.8 | 64.1 |

This measurement shows that Tg of the skim milk powder samples with increased GOS is clearly increased compared to regular lactose-free skim milk powder samples. An increase in Tg will lead to an improvement in drying properties and powder stability.

### EXAMPLE 5

### Determination of Water Sorption on Milk Powder Samples

A larger amount of dissolved skim milk powder was treated with BHT and Maxilact exactly as is described in Example 1 for Control A, Control C and Sample 16. A sample of each treated skim milk was dried on a laboratory spray drier (LabPlant SD-05, Keison Products) with an inlet air temperature of 128.8° C., an outlet air temperature of 78.1° C., an evaporation capacity of 1508.9 g/h, a skim milk flow rate of 1718.5 g/h and a powder flow rate of 176.5 g/h. 3 milligram of the skim milk powders produced using this method were analyzed in a dynamic vapour sorption machine (DVS—TA instruments Q5000sa) at 50° C. Moisture content (gram water/gram sample) was followed for 15 hours.

TABLE 7
Water sorption at 50° C. in time. Water
sorption is depicted as gram/gram sample

| Time (hr) | 5 | 10 | 15 |
|---|---|---|---|
| sample 16 | 0.014 | 0.031 | 0.050 |
| Control A | 0.011 | 0.023 | 0.038 |
| Control C | 0.015 | 0.035 | 0.056 |

The produced skim milk powders were stored and visually inspected at regular time intervals. The regular SMP (control A) did not show any visual clumping. while the LF-SMP (control C) showed severe clumping after a storage for more than one week. The BHT/Maxilact treated LF-SMP (sample 16) showed a clearly lower tendency to clump compared to control C.

1. A method for producing a lactose reduced dairy powder comprising
(i) treating a lactose comprising milk-based substrate with a first enzyme having transgalactosylase activity and a second enzyme having lactase activity to obtain a lactose reduced and galacto-oligosaccharides (GOS) comprising milk-based product
(ii) preparing lactose reduced dairy powder from said lactose reduced and GOS comprising milk-based product.

2. The method according to claim 1, wherein the milk-based product comprises at least 10% (w/w) GOS based on total carbohydrates.

3. The method according to claim 1, wherein the lactose in said milk-based product is reduced to 0.5 (w/v) %, optionally 0.1 (w/v) % and optionally <0.01 (w/v) % lactose.

4. The method according to claim 1, wherein the dairy powder comprises at least 4% (w/w) GOS.

5. The method according to claim 1, wherein the dairy powder comprises <5 (w/w) %, optionally <1 (w/w) % and optionally <0.1 (w/w) % lactose.

6. The method according to claim 1, wherein the enzyme having transgalactosylase activity is a Sporobolomyces singularis hexosyltransferase, a Bacillus circulans beta-galactosidase or a Bifidobacterium bifidum beta-galactosidase, optionally the enzyme having transgalactosylase activity is a Sporobolomyces singularis hexosyltransferase or a glycated Bacillus circulans beta-galactosidase and optionally the enzyme having transgalactosylase activity is a Sporobolomyces singularis hexosyltransferase.

7. The method according to claim 1, wherein the milk-based substrate has not been subjected to lactose reducing filtration technique.

8. The method according to claim 1, wherein the milk-based substrate is first treated with the enzyme having transgalactosylase activity and subsequently with the enzyme having lactase activity, optionally said method does not comprise an enzyme inactivation between treatment with the enzyme having transgalactosylase activity and treatment with the enzyme having lactase activity.

9. The method according to claim 1, wherein (i) is treating a lactose comprising milk-based substrate simultaneously with a first enzyme having transgalactosylase activity and a second enzyme having lactase activity to obtain a lactose reduced and galacto-oligosaccharides (GOS) comprising milk-based product.

10. The method according to claim 1, wherein said dairy powder is milk or whey powder.

11. Lactose free dairy powder comprising at least 4% (w/w) GOS.

12. The lactose free dairy powder according to claim 11 wherein the amount of lactose is <0.1 (w/w) % lactose.

13. Lactose free milk comprising at least 10% (w/w) GOS based on total carbohydrates and having a glucose/galactose ratio of >1.2.

14. Milk comprising <0.1% lactose, at least 10% GOS based on total (free/) carbohydrates and having a glucose/galactose ratio of >1.2.

15. The method according to claim 1, in which GOS improves drying of the lactose reduced milk-based substrate.

16. (canceled)

17. (canceled)

18. (canceled)


SEQUENCE LISTING (7 sequences)

SEQ ID NO: 1 - protein, 594 aa, Sporobolomyces singularis
  MISC_FEATURE (1)..(594): Beta-hexosyltransferase of organism Sporobolomyces singularis (also referred to as BHT-132, wildtype)
MMLHAALLVALPCVVLARPAGAVTYPGAIPLSLTSNYETPSPTAIPLEPTPTATGTAELDALWNLVEAQYPVQTAAVTTLVTVPDDYKFEADPPSYALAGYETSEIAGLKFPKGFKFGVAGAAIQVEGAAKAEGRGPSTWDYLCHHYASTQCNNYDPDITTNHYYLYPLDFARLQHLGINTYSFSISWTRIYPLGAGYVNEAGLAHYDAVIHSAKKYGLEPVGTVFHWDTPLSLMLKYGAWQDTGDQIVKDFVTYATTVFKRYGNEVKTWFTFNEPRVFCSQNSGLPYNLTYPEGINSTSAVFRCTYNVLKAHGHAVKVYRDLVASGTIAAGEIGFKSDDNYPIPARPGNADDEESAKRHEAFRIGIFAQPVYGNGDYPDVVKETVGDMLPALTDEDKGYIKGSGDIFAIDGYRTDISHAALNGIANCIRNQSDPNWPVCEEGSDPFAHVYPSGFAIGQSADPLSSWLVNSAPFIRDQLKFLTQTYPAKGGIYFSEFGWAEDAEYDRQLLYQITWDGLRTQYLTDYLSQLLLAVHKDGINLRGALTWSFVDNWEWGLGMQQKFGFQFVNQSDPDLTRTFKLSAHAYAQFGRNHL

SEQ ID NO: 2 - protein, 557 aa, Artificial Sequence
  sets out the sequence of BHT-134 which is a deletion mutant derived from SEQ ID NO 1 and the deletion starts at position 18 up to and including position 54
MMLHAALLVALPCVVLAGTAELDALWNLVEAQYPVQTAAVTTLVTVPDDYKFEADPPSYALAGYETSEIAGLKFPKGFKFGVAGAAIQVEGAAKAEGRGPSTWDYLCHHYASTQCNNYDPDITTNHYYLYPLDFARLQHLGINTYSFSISWTRIYPLGAGYVNEAGLAHYDAVIHSAKKYGLEPVGTVFHWDTPLSLMLKYGAWQDTGDQIVKDFVTYATTVFKRYGNEVKTWFTFNEPRVFCSQNSGLPYNLTYPEGINSTSAVFRCTYNVLKAHGHAVKVYRDLVASGTIAAGEIGFKSDDNYPIPARPGNADDEESAKRHEAFRIGIFAQPVYGNGDYPDVVKETVGDMLPALTDEDKGYIKGSGDIFAIDGYRTDISHAALNGIANCIRNQSDPNWPVCEEGSDPFAHVYPSGFAIGQSADPLSSWLVNSAPFIRDQLKFLTQTYPAKGGIYFSEFGWAEDAEYDRQLLYQITWDGLRTQYLTDYLSQLLLAVHKDGINLRGALTWSFVDNWEWGLGMQQKFGFQFVNQSDPDLTRTFKLSAHAYAQFGRNHL

SEQ ID NO: 3 - DNA, 1782 bp, Artificial Sequence
  sets out a codon-adapted DNA sequence for expression of SEQ ID NO 1 in Aspergillus niger
atgatgcttcacgctgctctccttgttgctcttccttgcgttgtccttgctcgtcccgccggtgctgtcacctaccccggtgccatccccctctccctcacctcgaactacgagactcccagccctactgccattcctctggagcccacccccactgccactggtactgctgagctcgatgccctctggaacttggttgaggctcagtaccccgtgcagactgctgccgtcaccaccctcgtcaccgttcccgatgactacaagttcgaggccgatcctcctagctacgctcttgctggatacgagacttctgagattgctggtctgaagttccccaagggcttcaagttcggtgttgctggtgccgccatccaggttgagggtgctgccaaggccgagggccgtggtccctccacctgggactacctctgccaccactacgcctccacccagtgcaacaactacgaccctgacatcaccaccaaccactactacctctaccccctcgacttcgctcgtctgcagcacctgggtatcaacacctactctttctccatctcctggactcgcatctaccctcttggtgctggctacgtcaacgaggctggtctggctcactacgatgccgtcatccactctgccaagaagtacggcttggagcctgttggtaccgtcttccactgggatactcctctgagcttgatgctcaagtacggtgcctggcaggacaccggtgaccagattgtcaaggacttcgtcacctacgccaccactgtcttcaagcgctacggcaacgaggtcaagacctggttcaccttcaacgagcctcgtgtcttctgctctcagaactccggtctgccctacaacctgacctacccagaaggcatcaactccacctcggcggtgttccgctgcacctacaacgtcctcaaggcccacggccacgccgtcaaggtctaccgtgatcttgttgcctccggtaccattgctgctggtgagatcggtttcaagtccgacgacaactaccccatccccgctcgccccggcaacgccgatgatgaggagtccgccaagcgtcacgaagcgttccgtatcggtatcttcgctcagcccgtgtatggcaacggtgactaccccgatgtcgtcaaggagactgttggtgacatgcttcccgccctcaccgacgaggacaagggctacatcaagggctccggtgacatcttcgccattgacggctaccgcactgatatatctcacgctgccctcaacggcattgccaactgcattcgcaaccagtccgaccccaactggcccgtctgcgaagaaggaagcgaccccttcgctcacgtctacccctccggattcgccatcggtcagtctgccgaccctctgagcagctggctggtcaactctgctcccttcatccgtgaccagctcaagttcctgacccagacctaccctgccaagggtggtatctacttctccgagttcggctgggcggaagatgcggaatacgaccgtcagctcctctaccagatcacctgggatggcctgcgcacccagtacctgaccgactacctcagccagctcctccttgccgttcacaaggatggcatcaacctgcgtggagcgttgacctggtcgttcgtcgacaactgggaatggggcttgggaatgcagcagaagttcggtttccagttcgtcaaccagtcggaccccgatctgacccgcaccttcaagctctccgcccacgcctacgctcagttcggccgcaaccacctg

SEQ ID NO: 4 - DNA, 1671 bp, Artificial Sequence
  sets out a codon-adapted DNA sequence for expression of SEQ ID NO 2 in Aspergillus niger
atgatgctccacgctgccttgcttgttgctctcccctgcgttgttctcgccggtactgctgagctcgatgccctctggaacctggttgaagcgcagtaccccgtgcagactgctgctgtcaccactctcgtcaccgtccccgatgactacaagttcgaggccgaccctcctagctacgcccttgctggctacgagacttctgagatcgctggtctgaagttccccaagggtttcaagttcggtgttgctggtgctgctatccaggttgagggtgctgccaaggccgagggccgtggtcccagcacctgggactacctctgccaccactacgccagcacccagtgcaacaactacgaccccgatatcaccaccaaccactactacctgtaccctctggacttcgctcgcttacaacacctgggcatcaacacctactctttctccatcagctggactcgtatctacccccttggtgctggatacgtcaacgaggctggtcttgctcactacgatgccgtcatccactccgccaagaagtacggcttggagcctgttggtaccgtcttccactgggatactcctctttctctgatgctcaagtacggtgcctggcaggacactggtgaccagattgtgaaggacttcgtcacctacgccaccaccgtcttcaagcgttacggcaacgaggtcaagacctggttcaccttcaacgagcctcgtgtcttctgctcgcagaactccggtctgccctacaacttgacttaccccgagggtatcaactccacctccgccgtcttccgctgcacctacaacgtcctcaaggcccacggccacgccgtcaaggtctaccgtgatcttgttgcctccggtaccattgctgccggtgaaattggattcaagtcggatgacaactaccccatccctgctcgccccggcaacgccgatgatgaggagtctgccaagcgtcacgaagcgttccgcattggtatcttcgctcagcctgtctacggtaacggtgactaccccgatgttgtcaaggagactgtcggtgacatgcttcctgctctgaccgacgaggacaagggctacatcaagggttccggtgacatcttcgccatcgacggctaccgcactgacatctcccacgctgctctcaacggcattgccaactgcattcgcaaccagtccgaccccaactggcccgtctgcgaagaaggctccgaccccttcgctcacgtctacccctccggcttcgccattggccagtccgccgaccccctcagcagctggctggtcaactctgctcccttcatccgtgaccagctcaagttcctgacccagacctaccccgccaagggtggtatctacttctctgagttcggctgggctgaggatgccgagtacgaccgccagctcctctaccagatcacctgggatggtctgcgtacccagtacctgaccgactacctgtcccagctcctgctggccgtccacaaggatggcatcaacctccgtggtgccctcacctggtcgttcgtcgacaactgggaatggggtcttggcatgcagcagaagttcggtttccagttcgtcaaccagtcggatcctgacctcacccgcaccttcaagctctccgctcacgcctacgcccagttcggccgcaaccattta

SEQ ID NO: 5 - DNA, 13 bp, Artificial Sequence
  sets out the modified translational initiation sequence of the glucoamylase glaA promoter
caccgtcaaaatg

SEQ ID NO: 6 - DNA, 1998 bp, Artificial Sequence
  sets out the DNA fragments containing a.o. part of the glucoamylase promoter and the BHT-132 encoding gene including an EcoRI and PacI restriction site
gaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacaccgtcaaaatgatgcttcacgctgctctccttgttgctcttccttgcgttgtccttgctcgtcccgccggtgctgtcacctaccccggtgccatccccctctccctcacctcgaactacgagactcccagccctactgccattcctctggagcccacccccactgccactggtactgctgagctcgatgccctctggaacttggttgaggctcagtaccccgtgcagactgctgccgtcaccaccctcgtcaccgttcccgatgactacaagttcgaggccgatcctcctagctacgctcttgctggatacgagacttctgagattgctggtctgaagttccccaagggcttcaagttcggtgttgctggtgccgccatccaggttgagggtgctgccaaggccgagggccgtggtccctccacctgggactacctctgccaccactacgcctccacccagtgcaacaactacgaccctgacatcaccaccaaccactactacctctaccccctcgacttcgctcgtctgcagcacctgggtatcaacacctactctttctccatctcctggactcgcatctaccctcttggtgctggctacgtcaacgaggctggtctggctcactacgatgccgtcatccactctgccaagaagtacggcttggagcctgttggtaccgtcttccactgggatactcctctgagcttgatgctcaagtacggtgcctggcaggacaccggtgaccagattgtcaaggacttcgtcacctacgccaccactgtcttcaagcgctacggcaacgaggtcaagacctggttcaccttcaacgagcctcgtgtcttctgctctcagaactccggtctgccctacaacctgacctacccagaaggcatcaactccacctcggcggtgttccgctgcacctacaacgtcctcaaggcccacggccacgccgtcaaggtctaccgtgatcttgttgcctccggtaccattgctgctggtgagatcggtttcaagtccgacgacaactaccccatccccgctcgccccggcaacgccgatgatgaggagtccgccaagcgtcacgaagcgttccgtatcggtatcttcgctcagcccgtgtatggcaacggtgactaccccgatgtcgtcaaggagactgttggtgacatgcttcccgccctcaccgacgaggacaagggctacatcaagggctccggtgacatcttcgccattgacggctaccgcactgatatatctcacgctgccctcaacggcattgccaactgcattcgcaaccagtccgaccccaactggcccgtctgcgaagaaggaagcgaccccttcgctcacgtctacccctccggattcgccatcggtcagtctgccgaccctctgagcagctggctggtcaactctgctcccttcatccgtgaccagctcaagttcctgacccagacctaccctgccaagggtggtatctacttctccgagttcggctgggcggaagatgcggaatacgaccgtcagctcctctaccagatcacctgggatggcctgcgcacccagtacctgaccgactacctcagccagctcctccttgccgttcacaaggatggcatcaacctgcgtggagcgttgacctggtcgttcgtcgacaactgggaatggggcttgggaatgcagcagaagttcggtttccagttcgtcaaccagtcggaccccgatctgacccgcaccttcaagctctccgcccacgcctacgctcagttcggccgcaaccacctgtaaattaattaa

SEQ ID NO: 7 - DNA, 1887 bp, Artificial Sequence
  sets out the DNA fragments containing a.o. part of the glucoamylase promoter and the BHT-134 encoding gene including an EcoRI and PacI restriction site
gaattcaagctagatgctaagcgatattgcatggcaatatgtgttgatgcatgtgcttcttccttcagcttcccctcgtgcagatgaggtttggctataaattgaagtggttggtcggggttccgtgaggggctgaagtgcttcctcccttttagacgcaactgagagcctgagcttcatccccagcatcattacaccgtcaaaatgatgctccacgctgccttgcttgttgctctcccctgcgttgttctcgccggtactgctgagctcgatgccctctggaacctggttgaagcgcagtaccccgtgcagactgctgctgtcaccactctcgtcaccgtccccgatgactacaagttcgaggccgaccctcctagctacgcccttgctggctacgagacttctgagatcgctggtctgaagttccccaagggtttcaagttcggtgttgctggtgctgctatccaggttgagggtgctgccaaggccgagggccgtggtcccagcacctgggactacctctgccaccactacgccagcacccagtgcaacaactacgaccccgatatcaccaccaaccactactacctgtaccctctggacttcgctcgcttacaacacctgggcatcaacacctactctttctccatcagctggactcgtatctacccccttggtgctggatacgtcaacgaggctggtcttgctcactacgatgccgtcatccactccgccaagaagtacggcttggagcctgttggtaccgtcttccactgggatactcctctttctctgatgctcaagtacggtgcctggcaggacactggtgaccagattgtgaaggacttcgtcacctacgccaccaccgtcttcaagcgttacggcaacgaggtcaagacctggttcaccttcaacgagcctcgtgtcttctgctcgcagaactccggtctgccctacaacttgacttaccccgagggtatcaactccacctccgccgtcttccgctgcacctacaacgtcctcaaggcccacggccacgccgtcaaggtctaccgtgatcttgttgcctccggtaccattgctgccggtgaaattggattcaagtcggatgacaactaccccatccctgctcgccccggcaacgccgatgatgaggagtctgccaagcgtcacgaagcgttccgcattggtatcttcgctcagcctgtctacggtaacggtgactaccccgatgttgtcaaggagactgtcggtgacatgcttcctgctctgaccgacgaggacaagggctacatcaagggttccggtgacatcttcgccatcgacggctaccgcactgacatctcccacgctgctctcaacggcattgccaactgcattcgcaaccagtccgaccccaactggcccgtctgcgaagaaggctccgaccccttcgctcacgtctacccctccggcttcgccattggccagtccgccgaccccctcagcagctggctggtcaactctgctcccttcatccgtgaccagctcaagttcctgacccagacctaccccgccaagggtggtatctacttctctgagttcggctgggctgaggatgccgagtacgaccgccagctcctctaccagatcacctgggatggtctgcgtacccagtacctgaccgactacctgtcccagctcctgctggccgtccacaaggatggcatcaacctccgtggtgccctcacctggtcgttcgtcgacaactgggaatggggtcttggcatgcagcagaagttcggtttccagttcgtcaaccagtcggatcctgacctcacccgcaccttcaagctctccgctcacgcctacgcccagttcggccgcaaccatttataaattaattaa